(19) United States
(12) Patent Application Publication
(10) Pub. No.: US 2023/0323488 A1
(43) Pub. Date: Oct. 12, 2023

(54) RAPID AND LOW-COST SAMPLING FOR DETECTION OF AIRBORNE SARS-COV-2 IN DEHUMIDIFIER CONDENSATE

(71) Applicants: University of Maryland, Baltimore County, Baltimore, MD (US); University of Maryland, Baltimore, Baltimore, MD (US)

(72) Inventors: Govind RAO, Ellicott City, MD (US); Xudong GE, Woodstock, MD (US); Douglas D. FREY, Ellicott City, MD (US); Sai Sathish RAMAMURTHY, Reisterstown, MD (US); Dipanjan PAN, Ellicott City, MD (US)

(21) Appl. No.: 18/298,540

(22) Filed: Apr. 11, 2023

Related U.S. Application Data
(63) Continuation of application No. PCT/US2021/054526, filed on Oct. 12, 2021.
(60) Provisional application No. 63/090,926, filed on Oct. 13, 2020.
(60) Provisional application No. 63/119,135, filed on Nov. 30, 2020.

(51) Int. Cl. C12Q 1/70; C12Q 1/6844; G01N 33/569; G01N 1/40; B01L 3/00; G01N 33/543

(52) U.S. Cl. CPC C12Q 1/701; C12Q 1/6844; G01N 33/56983; G01N 1/40; B01L 3/502761; G01N 33/54386; G01N 2333/165; B01L 2200/0652; B01L 2200/16

(57) ABSTRACT

The present invention relates to the use of a condensate collector such as portable or stationary dehumidifier placed in a defined testing space or area and used as a readily available and affordable tool to collect airborne virus particles in collected condensate from the testing atmosphere in the defined testing space or area, wherein the collected condensate is analyzed for virus biomarkers to identify viruses, such as SARS-CoV-2 or mutants or variants thereof, in the testing atmosphere.

### CROSS REFERENCE TO RELATED APPLICATION

This application is filed under the provisions of 35 U.S.C. § 120 and is a continuation-in-part of International Patent Application No. PCT/US2021/054526 filed on Oct. 12, 2021, which claims priority to U.S. Provisional Patent Application No. 63/090,926 filed on Oct. 13, 2020, and U.S. Provisional Patent Application No. 63/119,135 filed Nov. 30, 2020, the contents of which are all hereby incorporated by reference herein for all purposes.

### BACKGROUND OF THE INVENTION

### Technical Field

The present invention relates to the use of a condensate collector such as portable or stationary dehumidifier placed in a defined testing space or area and used as a readily available and affordable tool to collect airborne virus particles in collected condensate, wherein the collected condensate is analyzed for virus biomarkers, such as virus envelope proteins and/or RNA to identify viruses including, but not limited to SARS-CoV-2, in the testing atmosphere.

### Related Art

Since the emergence of the first case of coronavirus in Wuhan, China in December 2019, coronavirus disease 2019 (COVID-19) has infected millions of people and claimed over four (4) million lives worldwide with a staggering number of affected individuals in the United States. The unavailability of rapid testing has severely hampered efforts to manage the disease and assess its risk of transmission. Furthermore, uncertainty about its mode of spreading has created much perplexity and resulted in incoherent and constantly changing guidelines (Lewis, 2020), creating public confusion and noncompliance. The case of mass infections from the Biogen conference, the Washington Choir (Hamner, 2020) and the Wuhan restaurant (Lu et al., 2020) are concrete evidence of the ease with which social contact can spread the virus. The World Health Organization (WHO) has been evaluating the spread of severe acute respiratory syndrome coronavirus 2 (SARS-CoV-2) with the understanding that the infection transmission mode is primarily respiratory through airborne transmission of aerosols (Prather et al., 2020). Due to the shared similarities between SARS-CoV-2 and other coronaviruses like the Middle East respiratory syndrome (MERS-CoV) and severe acute respiratory syndrome (SARS-CoV), both of which were found to be airborne and could be potentially transmitted to long distances, it is essential to investigate this feature of the new virus and mitigate any plausible risks. Based on aerodynamic analyses in hospital settings, there is growing evidence for airborne transmission of COVID-19 that causes resurgence of infection in closed network topology (Chia et al., 2020; Guo et al., 2020; Liu et al., 2020; Ma et al., 2020; Morawska & Cao, 2020; Morawska, Tang, Bahnfleth, et al., 2020; Santarpia,] Herrera, et al., 2020; Santarpia, Rivera, et al., 2020; Stadnytskyi et al., 2020). Recent findings from a study conducted in a hospital ward further confirmed aerosol-based transmission of viable SARS-CoV-2 from air samples collected 2-4.8 m away from patients (Lednicky et al., 2020). This necessitates detection protocols to be in place for modeling strategic quarantining policies and to assess transmission dynamics. Hence, understanding of exposure risk to these lethal bioaerosols is vital to implement near real-time interventions to prevent the spread of the virus as well as safeguard human health (Prussin & Marr, 2015). This is especially useful, as several reports indicating the spread of the virus through asymptomatic and presymptomatic patients have surfaced (Furukawa et al., 2020; Lee et al., 2020). For example, asymptomatic carriers are believed to be major spreaders and the virus efficiently moves on droplets from infected people. It is believed that a mere 1,000 virus inoculum may be sufficient to infect someone. For reference, a sneeze/cough from an infected person is believed to eject millions of viruses into the atmosphere, while speaking is believed to eject several thousand particles per minute.

However, easily implemented approaches to assess the actual environmental threat are currently unavailable. As such, there is a need for a simple, robust method, capable of providing rapid and accurate results on virus exposure such as SARS-CoV-2 to slow the spread of the disease and cater to community health at large.

### SUMMARY OF THE INVENTION

The present invention provides for a method of collecting bioaerosols particles in a defined area, wherein the bioaerosols particles are collected in the condensate of a condensate collector positioned in the defined area, and collected condensate is analyzed to isolated bioaerosols particles indicative of virus biomarkers.

In one aspect, the present invention provides for a method of collecting bioaerosols virus particles in a defined area, wherein the aerosols virus particles are collected in the condensate of a dehumidifier positioned in the defined area, isolated and analyzed for coronavirus biomarkers, including COVID-19 and mutants or variants thereof.

In another aspect, the present invention provides for a system for detecting aerosolized virus particles or biomarkers in an atmosphere within a defined space, the system comprising:
a dehumidifier for collecting the aerosolized virus particles or biomarkers, wherein collected aerosolized virus particles or biomarkers are contained in the condensate of the dehumidifier;
a collection system for removing the condensate from the dehumidifier;
a device for concentrating collected aerosolized virus particles or biomarkers, wherein the collection system is communicatively connected to the affinity microcolumn; and a detection system for analyzing the condensate.

In a preferred embodiment system, the dehumidifier is a low-grain refrigerant (LGR) dehumidifier, the device for concentration is an affinity microcolumn and the detection system uses Rapid flow enzyme-linked immunosorbent assay (ELISA) detection. Preferably, testing is conducted in an area under humidity conditions ranging from 40-60% at room temperature of about 25° C. Importantly and preferably the LGR unit is able to process about 4 cubic meters air per minute (CMM) or about 4,000 liters per minute for optimal testing.

In yet another aspect, the present invention provides for a method of detecting aerosolized virus particles or biomarkers in an atmosphere within a defined space, the method comprising:
positioning a dehumidifier in the defined space and collecting any aerosolized virus particles or biomarkers in the atmosphere for about 10 minutes to several days, preferably from min to 24 hours;
removing the condensate from the dehumidifier;
concentrating the condensate to isolate any captured virus particles or biomarkers in the condensate thereby forming a concentrated solution of virus particles or biomarkers;
analyzing the concentrated solution of virus particles or biomarkers to determine virus type and quantity.

Optionally, if the atmosphere does not include a sufficient content of moisture, a humidifier can be added to the defined space which will increase humidity for better dehumidifier operation.

Analyzing the samples may include but is not limited to a protein enzyme-linked immunosorbent assay (ELISA) kit or aliquoted and freeze-dried for RNA-based analysis employing commercially available RT-LAMP and reverse-transcription polymerase chain reaction (RT-PCR) kits. As such, an ELISA assay for targeting towards SARS-CoV-2 spike (S) protein is effective and designed primers for RT-LAMP and RT-PCR assays to target a SARS-CoV-2 genes. Another detection technique includes a nano-sensing platform from lanthanide-doped carbon nanoparticles (LCNPs) to provide a distinct fluorescence response in presence of SARS-CoV-2.

In a further aspect, the present invention provides for implementing a dehumidifier that is placed in areas of high footfall as an effective way of collecting virus particles and thus controlling the spread of a deadly virus disease, such as a Coronavirus or mutants or variants thereof, and provides a method of bypassing individual testing on a continuous basis. If the area is a hospital ward and testing of the condensate from the dehumidifier found that the virus particle count is non-existent, then there is a reduced need for individual testing. The present invention that analyzes condensate collected from the atmosphere in a dehumidifier provides a simple and effective means of assessing viral load in the defined surroundings. Additionally, camera surveillance could be helpful in identifying subjects in the defined space during the testing period.

In another aspect, the present invention further provides for testing the condensate collected from the dehumidifier and analyzing for RNA or virus particles, including protein of the virus such as the S-protein from COVID-19 or mutants or variants thereof.

In another aspect, it is beneficial to include Viral transport medium (VTM) in the dehumidifier chamber to reduce the possibility of viral destabilization in the sampling method and to ensure the stability of any viral RNA in the condensate.

In a still further aspect, the present invention provides for the use of condensate collector for detecting aerosolized virus particles or biomarkers in an atmosphere within a defined space comprising:
positioning a dehumidifier in the defined space and collecting any aerosolized virus particles or biomarkers in the atmosphere for about 10 minutes to several days, preferably from min to 24 hours;
removing the condensate from the dehumidifier;
concentrating the condensate to isolate any captured virus particles or biomarkers in the condensate thereby forming a concentrated solution of virus particles or biomarkers;
analyzing the concentrated solution of virus particles or biomarkers to determine virus type and quantity.

Notably, the present invention preferably uses a condensation system similar to a dehumidifier but any method that extracts moisture from atmospheric air may be used to produce a condensate, such as compressing the sampled air and rapidly expanding it through a nozzle, resulting in adiabatic cooling that makes the moisture condense. Further, other systems may be considered, such as a water harvester system that use a metallic organic framework to capture water molecules, an atmospheric water generator (AWG) that extracts water from ambient air or a peltier cooler.

Additional features and advantages of the subject matter of the present disclosure will be set forth in the detailed description which follows, and in part will be readily apparent to those skilled in the art from that description or recognized by practicing the subject matter of the present disclosure as described herein, including the detailed description which follows, the claims, as well as the appended figures.

### BRIEF DESCRIPTION OF FIGURES

FIG. 1 A is a schematic representation of sample collection and analysis for mass detection.

FIG. 1 B shows a simplified layout of the hospital ward indicating the positions of various dehumidifiers.

FIG. 1C shows another view of the placement of various dehumidifiers during the testing examples.

FIG. 2 shows the concentration of severe acute respiratory syndrome coronavirus 2 (SARS-CoV-2 S)-protein as determined by the enzyme-linked immunosorbent assay (ELISA) for the samples collected. The sample code starts with the date of sample collection from the hospital followed by the dehumidifier number, that is, 0630_4 indicates the water sample has been collected from dehumidifier number 4 on the respective dates.

FIG. 3 shows the determination of SARS-CoV-2 S-protein concentration using protein-based ELISA assay. Again, the sample code starts with the date of sample collection from the hospital followed by the dehumidifier number, that is, 0910_1 b indicates the water sample has been collected from dehumidifier number 1 in a different time period.

FIG. 4 shows a plot of conductivity (blue line) and humidity (red line) inside the test room. After leaving the dehumidifier on overnight, the humidifier was turned on (vertical orange line). Two hours later, 10 gm sodium chloride was added to the water inside the humidifier (vertical green line). Six hours after the addition of salt, the humidifier was removed from the room (dashed vertical purple line). Over the course of the experiment, the temperature was maintained at 22±3° C. Photos showing humidifier, dehumidifier and conductivity meter are included.

FIG. 5 shows the standard curve for the determination of concentration of SARS-CoV-2 S-protein.

FIG. 6 shows the increase in emission intensity at 520 nm post RT-LAMP reaction for the BEI gamma-killed virus samples spiked with condensate in VTM.

FIG. 7 shows a system with integrated hardware to condense, capture and detect any airborne virus particles or biomarkers.

### DETAILED DESCRIPTION OF THE INVENTION

Various embodiments of the disclosure will be described in detail with reference to figures. Reference to various embodiments does not limit the scope of the invention. Additionally, any examples set forth in this specification are not limiting and merely set forth some of the many possible embodiments of the claimed invention.

All scientific and technical terms used herein have meanings commonly used in the art unless otherwise specified. The definitions provided herein are to facilitate understanding of certain terms used frequently herein and are not meant to limit the scope of the present disclosure.

“Include,” “includes,” or like terms means encompassing but not limited to, that is, inclusive and not exclusive.

“Optional” or “optionally” means that the subsequently described step, feature, condition, characteristic, or structure, occurs/is present or does not occur/is not present, while still being within the scope described.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the inventive technology.

As used in this specification and the appended claims, the term “or” is generally employed in its sense including “and/or” unless the content clearly dictates otherwise. The term “and/or” means one or all of the listed elements or a combination of any two or more of the listed elements.

As used herein, “have”, “has”, “having”, “include”, “includes”, “including”, “comprise”, “comprises”, “comprising” or the like are used in their open-ended inclusive sense, and generally mean “include, but not limited to”, “includes, but not limited to”, or “including, but not limited to”.

While various features, elements or steps of particular embodiments may be disclosed using the transitional phrase “comprising,” it is to be understood that alternative embodiments, including those that may be described using the transitional phrases “consisting of” or “consisting essentially of” are implied.

“Spike protein” or “S” protein as used interchangeably herein refers to one of four main structural proteins of a coronavirus. The spike protein is heavily N-linked glycosylated and utilizes an N-terminal signal sequence to gain access to the endoplasmic reticulum (ER). Homotrimers of the virus-encoding S protein make up the distinctive spike structure on the surface of the virus. In many coronaviruses, the S protein is cleaved by a host cell furin-like protease into two separate polypeptides noted S1 and S2. S1 makes up the large receptor-binding domain (RBD) of the S protein while S2 forms the stalk of the spike molecule.

As previously stated, airborne spread of coronavirus disease 2019 (COVID-19) by infectious aerosol is currently spreading around the globe. The present invention provides a novel approach to rapidly provide information about the prevalence of a virus, for example an aerosolized virus such as a coronavirus, e.g., severe acute respiratory syndrome coronavirus 2 (SARS-CoV-2), in the atmosphere at any location. The use of a portable or stationary dehumidifier is an available and affordable tool to collect airborne virus in the condensate. The dehumidifiers are deployed in selected locations of a defined space, such as a hospital ward with patients reporting flu-like symptoms which could possibly be due to a coronavirus, e.g., COVID-19, and testing of the area for aerosols particles can be easily monitored. Samples are then analyzed frequently for virus biomarkers, such as virus envelope protein and SARS-CoV-2 RNA. The present invention provides a facile pool testing method to sample air in any location in the world and assess the presence and concentration of an infectious agent to obtain quantitative risk assessment of exposure, designate zones as “hot spots” and minimize the need for individual testing which may often be time consuming, expensive, and laborious.

For the testing methods of the present invention, a condensate collector of atmospheric particles or virus biomarkers is placed in an area to monitor such atmospheric particles or virus biomarkers for a specific virus. Any such condensate collector device, such as a portable or stationary dehumidifier may be used. The dehumidifier can be either a conventional dehumidifier or more preferably a low-grain refrigerant (LGR) dehumidifier because LGR dehumidifiers provide maximum power in removing moisture from the air, LGR dehumidifiers have a double cooling system that lowers the temperature of moisture-filled air once inside the machine. This leads to more condensation that can be easily collected for further testing of virus particles. Preferably, testing is conducted in an area under humidity conditions ranging from 40-60% at room temperature of 25° C., which is a typical parameter for conditioned space. Importantly and preferably the LGR unit is able to process 4 cubic meters air per minute (CMM) or 4,000 liters per minute for optimal testing.

As air is drawn through a LGR dehumidifier it is basically a two-stage system capable of achieving very efficient condensation. The rated condensate generation capacity is approximately 1.5 liter/hour, so one can expect 50 mL condensate collected in two minutes. As stated above, the collection efficiency is calibrated in an environmental chamber under humidity conditions ranging from 40-60% at room temperature of 25° C., which is a typical parameter for conditioned space. Ideally, the unit is able to process 4 cubic meters air per minute (CMM) or 4,000 liters per minute. Notably, conventional aerosol samplers cannot achieve these rates unless scaled up to impractical sizes.

Testing of the collected condensate from the dehumidifier may include several testing methods to determine the viral load including chromatographic capture of the virus and then using a rapid ELISA for quantitation. By deploying an LGR dehumidifier that can pull in 4,000 L of air in one minute and generate 50 mL condensate, about 8,000 ug over can be captured in about two minutes. Assuming 5 mL of this binds to a 25-microliter capture column in 10 minutes and a 1 column volume elution is performed capture could be in the range of 32 pg/mL Other sensitive detection schemes may include systems such as I-dimensional photonic crystal/Bragg grating based monitoring of the fluorescent immunoassay or biosensing with fluorescent nanodiamonds.

The target area for testing is achieved by a simple sizing of the dehumidifier used, or by simply deploying additional units. The present invention considers that conventional bioaerosol samplers may not be as effective although having higher collection efficiency because they rely on impingement/membranes for collection and so would require enormous samplers (or very long times) to achieve the same sample rate that is needed.

### Examples

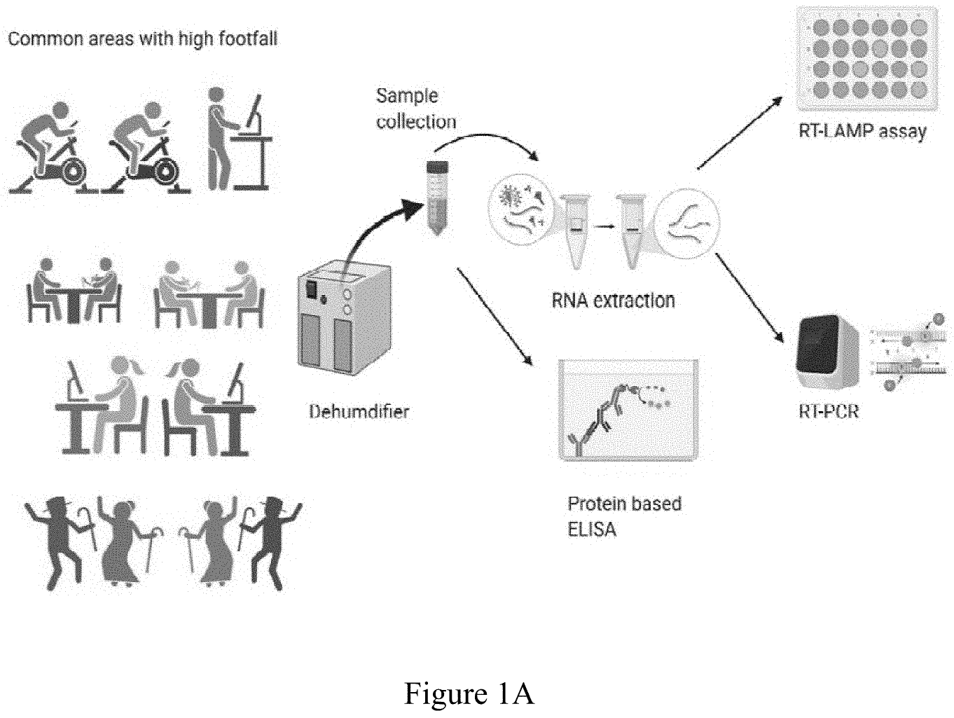

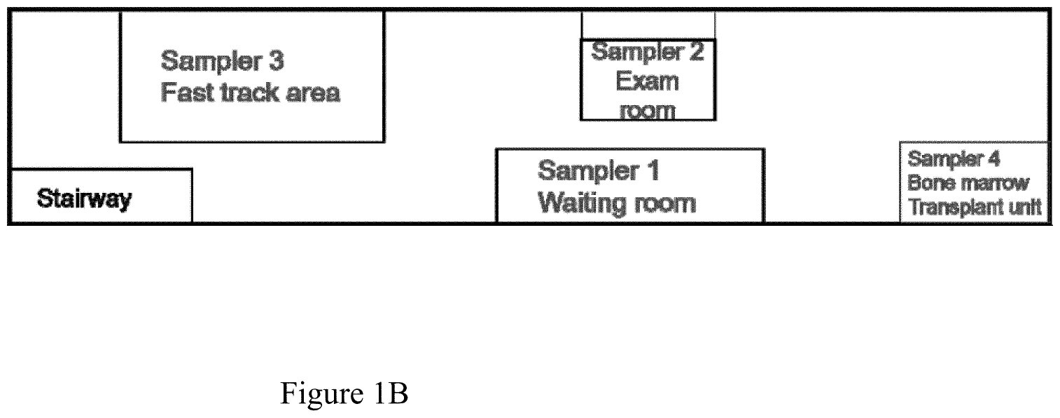

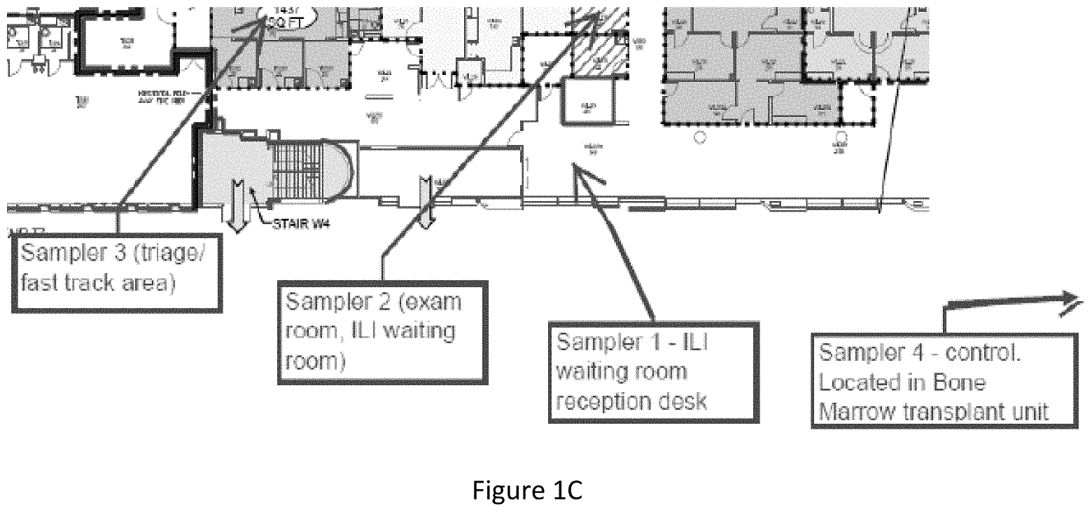

For testing, four portable dehumidifiers were placed at various test locations around a hospital ward at the University of Maryland Medical Center in Baltimore and such dehumidifiers obtained condensate samples for viral load analysis on different dates. The systems and placement are shown in FIGS. 1A, B and C. The condensate was sampled at three separate time periods, each lasting about a week and testing times about two weeks apart. Viral transport medium (VTM) liquid Amies from Innovative Research was used for this study. This is a clear, colorless liquid and is negative for microbial growth after 48 h at 37° C. The VTM consists of 1× sterile Hanks balanced salt solution (HBSS) with calcium and magnesium ions, 2% heat-inactivated fetal bovine serum, gentamicin sulfate (100 μg/ml) and amphotericin B (0.5 μg/ml).

The present air sampling methodology is a robust indicator of a potential contact pool of SARS-CoV-2 which can be used as a tool to implement strategies in a community bubble. Furthermore, it helps in developing isolation strategies focusing on reducing disease burden thereby lowering morbidity and mortality. Thus, community mixing can be restricted through various social behavioral patterns by indirectly measuring the surge in COVID-19 as opposed to observing confirmed cases (La Rosa et al., 2020), many of which could have been arrested beforehand. By employing this simple methodology, as shown in FIG. 1A to monitor the presence of SARS-CoV-2 or other mutant or variant viruses especially in areas with high human footfall or mass gatherings, appropriate preventive measures can be adopted to identify, track possible hotspots, and protect individuals from being infected. Among other uses, this type of monitoring can be used to enhance the effectiveness of vaccination strategies.

### Materials and Methods

Four identical 900 ml dehumidifiers (ICETEK B0863HNVNS from Amazon) were numbered 1-4 and deployed at the various sites indicated in FIGS. 1 B and C. These dehumidifiers used a muffin fan that draws room air past a Peltier-cooled heat exchanger and deposits condensate into a tank underneath. While one dehumidifier was placed in the command center of the hospital to serve as a control, the other dehumidifiers were placed in staging areas involving the use of automated external defibrillators and powered air-purifying respirator units. The condensate tanks were sampled at 24 or 48 h intervals and 50 ml samples were further processed for analysis. A three-prong approach was followed for the identification of viral load in the collected condensate samples. All samples were deactivated in a water bath set at 65° C. for 30 min, following which they were either stored at 4° C. for protein detection using a protein enzyme-linked immunosorbent assay (ELISA) kit or aliquoted and freeze-dried for RNA-based analysis employing commercially available RT-LAMP and reverse-transcription polymerase chain reaction (RT-PCR) kits. It may be noted that the general targets towards the specific detection of SARS-CoV-2 are either the spike (S) or nucleocapsid (N) protein. As such, an ELISA assay was used for targeting towards SARS-CoV-2 spike (S) protein and also primers were designed for RT-LAMP and RT-PCR assay targeted towards SARS-CoV-2 N gene. Sampling lag, heat treatment, and 4° C. storage time may well have impaired stability of collected biomarkers and hence the added consideration for detection of S-protein and N-gene. As a parallel third detection technique, a previously developed nano-sensing platform from lanthanide-doped carbon nanoparticles (LCNPs) was employed which provided a distinct fluorescence response in presence of SARS-CoV-2 (Alafeef et al., 2019, 2020; Moitra et al., 2020). For samples collected in VTM, 50 ml of each sample was freeze dried and the residue redispersed in 2 ml of RNase free water and analyzed for the presence of RNA.

### Detection of Spike Protein (S-Protein) Using Commercial ELISA Kit.

The collected condensate samples were analyzed with a SARS-CoV-2 S-protein ELISA kit purchased from RayBiotech. This kit determined the presence and estimate of Spike protein (S2 subunit) of SARS-CoV-2 in the samples and was used as per manufacturer's protocol. Briefly, the ELISA technique was performed using a 96-well plate. Seven known concentrations (2000, 666.7, 222.2, 74.07, 24.69, 8.231, 2.744 ng/ml) of S-protein and 31 water samples) with unknown S-protein concentration were pipetted into the microliter plate wells with a volume of 100 μL of each sample. The plates were covered and incubated at room temperature (18-25° C.) for 2.5 h. After 2.5 h the wells were emptied, washed with diluted wash buffer, followed by addition of dilute biotinylated antibody (100 μL) and incubated for 1 h at room temperature. Wells were washed properly to eliminate the possibility of erroneous results. After 1 h, wells were washed again with diluted wash buffer, 100 μL streptavidin added to them and incubated for another 45 min at room temperature. After 45 mins the streptavidin solution was discarded, wells were washed properly, and 100 μL of the given substrate solution mixture was added. The well plate was covered and incubated for 30 mins at room temperature in the dark with gentle shaking. After 30 mins, stop solution was added to each well. The stop solution changes the color from blue to yellow, and the intensity (absorbance) of the color was measured at 450 nm using a BioTeK plate reader and Gen 5.0 software. Measurements were tested in duplicate sets, and the average value was then utilized to determine the final S-protein concentration.

### Extraction of RNA and Determination for the Presence of SARS-CoV-2 Using a Commercial One Step RT-qPCR Kit.

For RNA extraction, 50 mL of each heat inactivated water sample was aliquoted into sterile tubes, snap frozen and freeze dried (Freeze One 2.5, Labconco). Samples were then resuspended in 500 μL of sterile RNase free water, briefly vortexed and centrifuged at room temperature at 4000×g for 5 minutes. They were then lysed with an equal volume of lysis buffer containing 2-mercaptoethanol and an equal volume of 100% ethanol, vortexed and added to microcentrifuge tubes fitted with spin cartridges. The samples were centrifuged at and the flow through was discarded. They were then washed using wash buffer I and centrifuged again and washed twice with wash buffer II containing ethanol for the same time at the same speed following the manufacture's protocol (Invitrogen). All flow through was discarded and 200 μL of RNase free water was added to each sample and incubated for one minute at room temperature following which they were centrifuged for a few minutes and the eluent was collected. The eluent was analyzed using a NanoDrop (ThermoScientific) instrument for the presence of RNA and determination of its concentration.

If RNA was found to be present in the samples by Nanodrop measurements, then RT-PCR and RT-LAMP assay was performed to confirm the presence of SARS-CoV-2 virus, SARS-CoV-2. Accordingly, RT-PCR for SARS-CoV-2 detection was carried out using a One Step RT-qPCR Kit from GoldBio (St. Louis, MO) run on a StepOne Plus Real Time PCR instrument (Applied BioSystems, USA). The PCR cyclic process was performed using the program detailed in the RT-qPCR protocol kit. The CDC recommended probes were used for the assay: 2019-nCoV_N1 probe, 2019-nCoV_N2 probe. TaqMan® probes are labeled at the 5′-end with the reporter molecule 6-carboxyfluorescein (FAM) and with the quencher, Black Hole Quencher 1 (BHQ-1) at the 3′-end. Briefly, 5 μL of each RNA sample was treated with 1.5 probe mix, 10 μL of 2×Master Mix and the volume made up to 20 μL with RNase free water. The RT-PCR was run using a first cycling step of 30 minutes at 42° C. followed by one cycle of initial denaturation with a holding time of 3 minutes at 95° C. 40 cycles were used for denaturation and annealing/extension, the first with a holding time of 10 secs at 95° C. and the latter with a holding time of 30 seconds at 55° C. Data was analyzed thereafter.

### One-Step Loop-Mediated Isothermal Amplification (LAMP) of RNA Samples.

One-step Loop-Mediated Isothermal Amplification (LAMP) of RNA (RT-LAMP) targets was performed using WarmStart LAMP Kit (DNA & RNA) from New England BioLabs following the manufacturer's protocol. The primers were designed by PrimerExplorer V5 software targeted for N gene segment of SARS-CoV-2. Briefly, the primer mix was prepared and 5 of each RNA sample was treated with 12.5 μL of the supplied 2×Master Mix, 0.5 μL of fluorescent dye (50×), 2.5 μL of the prepared primer mix (10×) and the volume made up to 25 μL with RNase free water. The samples were then incubated on a heat block with gentle shaking at 65° C. for 30 minutes followed by deactivation at 85° C. for another 5 minutes. They were then diluted 3 times and added to the wells of a microplate reader and the fluorescent emission intensity was recorded.

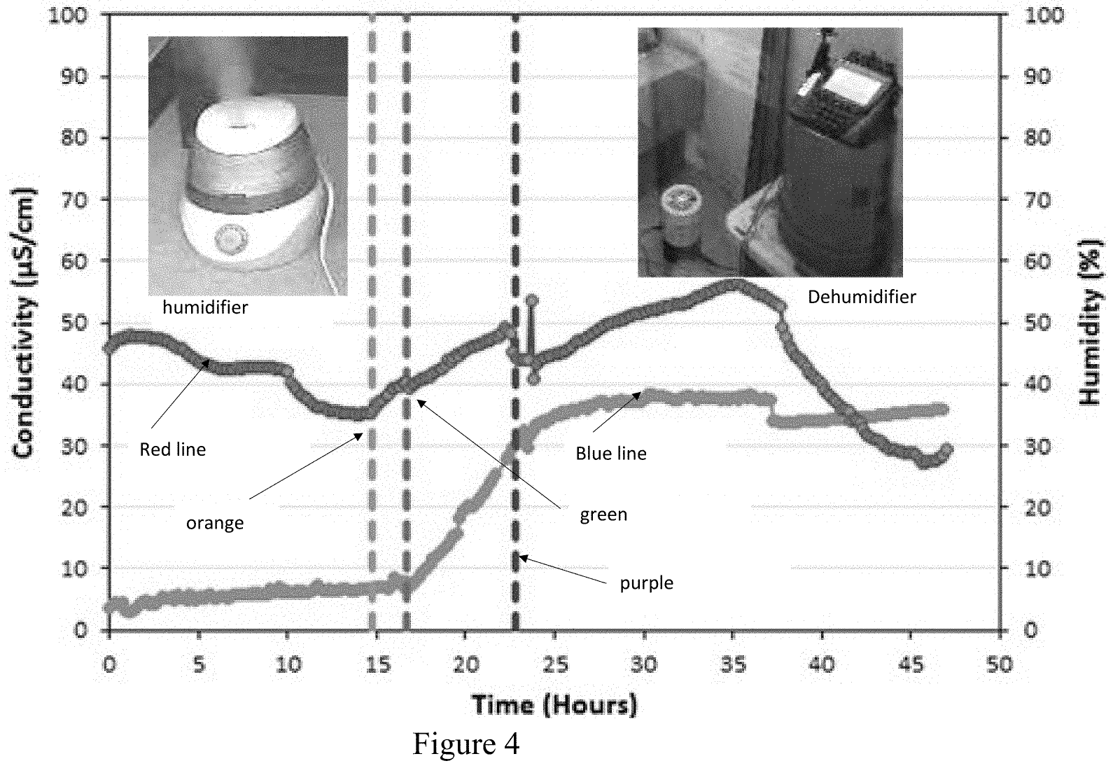

To further validate the ability of a dehumidifier to concentrate an aerosolized substance from the air, a cool-mist humidifier (CVS Health) and an 1800 cubic feet dehumidifier (ICETEK) were first placed inside a small, sealed room. After the operation of only the dehumidifier overnight, the humidifier was turned on (see FIG. 4). After 2 h, 10 g of sodium chloride was added to the water inside the humidifier. Six hours later, the humidifier was turned off and removed from the room. Over the course of the entire experiment, a GSP-6 temperature and humidity data logger (Elitech) were used to measure the humidity and temperature of the room and an Orion DuraProbe 4-electrode conductivity probe (Thermo Fisher Scientific) connected to an Orion VersaStar Pro electrochemistry meter (Thermo Fisher Scientific) was used to measure the conductivity of the condensate collected inside the dehumidifier. The temperature was maintained at 22±3° C.

### Results and Discussion

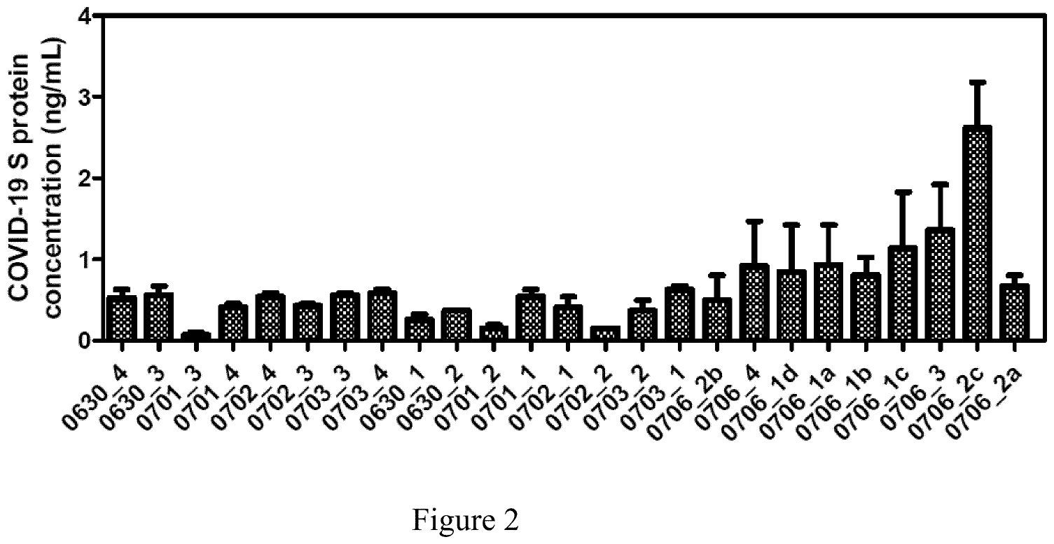

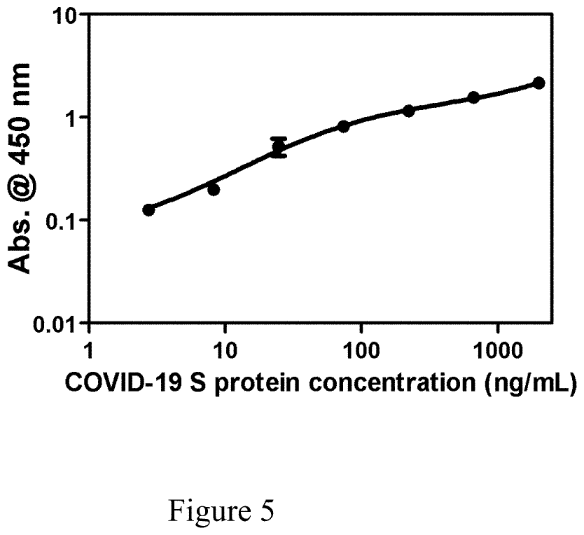

Water samples collected at the University of Maryland Medical Center were first analyzed using a SARS-CoV-2 S-protein ELISA kit. All the experiments were carried out at room temperature and samples were tested in duplicates. The mean value obtained was then utilized to determine the final S-protein concentration. A calibration curve was initially generated using the known S-protein concentrations (FIG. 5) and S-protein in the samples was then estimated (Table 2 below and FIG. 2). Only one sample presented a detectable dose of the virus SARS-CoV-2 S-protein while others were well below the detection limit of the kit. Interestingly, the virus could be detected only when sampling was continued over the weekend in comparison with the daily sampling protocol. This indicated the requirement of concentrate sampling for the successful detection of the viral spike protein.

A nanosensing platform that was previously developed in the inventor's laboratory was used as a parallel detection technique (Alafeef et al., 2019, 2020; Moitra et al., 2020). This platform was applied to 17 condensate (water) samples collected as described previously. The sensor consists of lanthanide-doped carbon nanoparticles (LCNPs) that provide a distinct fluorescence response towards the presence of SARS-CoV-2 specific viral protein (Table 3 below). The fluorescence responses obtained from the sensors were classified using a k-mean clustering machine-learning algorithm to identify the presence of SARS CoV-2 (Alafeef et al., 2019). The clustered signature attributes were used to identify the pathogen type based on the commonalities in the data set (Moitra et al., 2017). The results obtained were also compared with those from the ELISA kit to confirm the reliability of the lanthanide sensor matrix.

Based on the promising results just described above, an attempt was made to quantify the viral SARS-CoV-2 RNA in the water samples. Accordingly, RNA was extracted from all the samples (Table 4 below) and RT-LAMP and RT-PCR were performed to detect the presence of the viral SARS-CoV-2 RNA results obtained for the water samples indicated that no viral RNA was detected. This was attributed to either the low detection limit of the methods used or to deactivation or destabilization of SARS CoV-2 RNA in the dehumidifier chamber. To remove the possibility of viral destabilization in the sampling method, 50 ml VTM was added to the dehumidifier chamber. This was done to ensure the stability of the viral RNA in the condensate.

Following sample collection in VTM, and although RNA was detected in most of the samples (Table 5 below), both RT-PCR and RT-LAMP again did not detect viral RNA. Since sampling was performed at regular intervals and the condensate was collected as a whole instead of as fractions, this implies the presence of other detected RNAs alongside the viral RNA. Since VTM stabilizes RNA, it was inferred that there was cohabitation of all RNA types in the sampling chamber. This indicates that destabilization of viral RNA in the sampler is not the cause of the lack of viral RNA detection, but instead the cause is likely due to the relatively low sensitivity of the detection method used. To confirm this, an experiment was performed using gamma-killed virions from BEI (sample NR-52287, BEI Resources, NIAID, NIH, consists of a crude preparation of cell lysate and supernatant from Cercopithecus aethiops kidney epithelial cells (Vero E6; ATCC CRL-1586) infected with SARS-CoV-2, isolate USA-WA1/2020 that was gamma-irradiated (5×10⁶ RADs) on dry ice. The viral samples were diluted to similar concentrations as used for other samples obtained from the dehumidifier condensate. Two different concentrations were used and were spiked into the dehumidifier condensate. An RT-LAMP experiment was then performed using these samples which showed an increase in emission at 520 nm confirming the presence of SARS-CoV-2 viral RNA (FIG. 5). Hence, the discrepancy of RT-LAMP results among the spiked and actual dehumidifier condensate samples is likely attributed to the extensive dilution of the SARS-CoV-2 virus beyond the detection limit (0.75 copies/μL) of the RT-LAMP assay used in case of the actual samples.

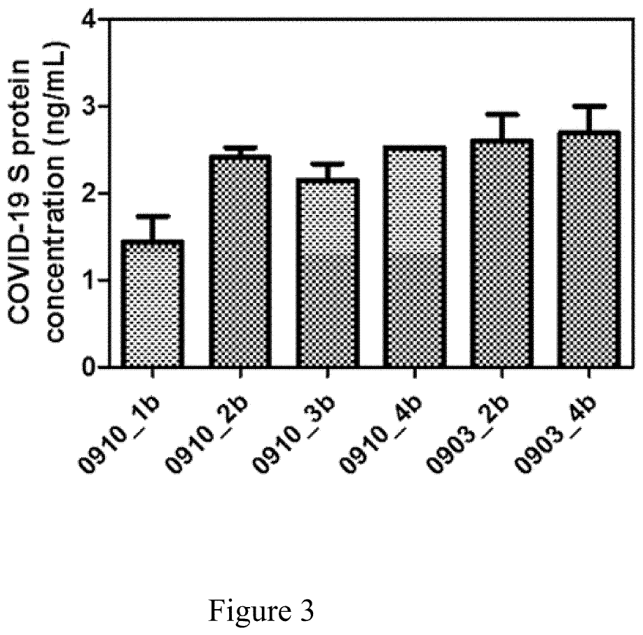

Interestingly, the presence of S-protein was still able to be detected close to the minimum detectable limit (FIG. 3). The sensitivity of the ELISA kit used (Protein ELISA from RayBiotech) is relatively lower than some of the recently available S-protein based ELISA kits. However, at the time of conducting this study, which was in the early stages of the pandemic, the only commercially available kit was from RayBiotech. This kit, which was the one employed in this study, had a detection range of 2.7-2000 ng/ml and therefore a relatively low sensitivity of 2.7 ng/ml.

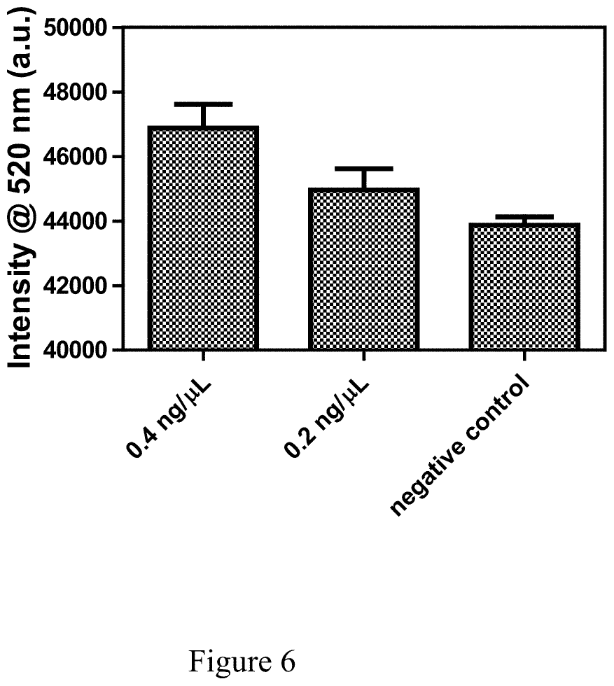

To further confirm the results, RT-PCR, RT-LAMP, and protein ELISA assays were performed with respect to positive and negative controls. For RT-PCR and RT-LAMP, quantitative PCR (qPCR) control RNA from heat-inactivated SARS-related coronavirus 2, isolate USA-WA1/2020, NR 52347, obtained from BEI, was used as the positive control and RNAse free water was used as the negative control. For the protein ELISA assay, the SARS-CoV-2 spike protein was used and provided with the kit as the positive control and assay buffer as the negative control. The standard curve, shown in FIG. 6, was generated accordingly with the kit provided S-protein. Thus, the positive results from the protein samples can be concluded to be true positives.

The present invention may be used in testing with different protein loads and contaminants generally encountered in a hospital environment. These samples will be aerosolized to assess interferences and to obtain data on false positives and negatives. Based on these studies, controls will be developed that will be used to assess accuracy and quantify false alarm rates. Also additional AI systems will be used and specifically Physics-Informed Neural Networks (PINNs) (Raissi et al., 2019) and deep learning methods for error detection and standardization of the sampling protocol.

Furthermore, it can be expected that the risk of false positives and negatives depends upon a variety of diverse factors. For example, if the kit used to perform either ELISA or RT-PCR is not sensitive enough towards the target, then a false negative may occur. Pekosz et al. (2021), recently conducted a study that evaluated both RT-PCR and antigen-based COVID-19 diagnosis using the conventional gold standard technique (i.e., virus culture in VeroE6TMPRSS2 cell). The study revealed that the antigen test demonstrated a higher positive predictive value (90%) than RT-PCR (70%) when compared with the virus culture results. The results found herein supports the antigen tests over RT-PCR. Therefore, the choice of the kit can affect both sensitivity and specificity of the obtained results. In addition, if the RT-PCR or ELISA technique is not performed following good molecular biology practices, carryover contamination might be observed in subsequent reactions resulting in false positive or false-negative results.

The overall results of this study are summarized in Table 1. Most strikingly, SARS-CoV-2 viral protein was detected over some period in all the samplers. This has implications for the efficacy of air filtration systems currently employed. Although airborne SARS-CoV-2 is widely implicated in the spread of COVID-19, there is great uncertainty over the precise mechanisms of exposure and susceptibility. The viral load in the atmosphere presumably fluctuates depending on the actual shedding by the infected persons and their number. The results cast a new light on this subject. However, the present invention has shown that the novel technique of sampling condensate from a dehumidifier can provide evidence of the airborne virus. Given the widespread use of air-conditioning equipment in homes and businesses worldwide, sampling their condensate provides a simple means of pool testing for virus presence analogous to those proposed for sewage monitoring. This approach also solves the major problem faced by conventional swab or saliva testing, where results can take several days. Antibody and point-of-care (POC) tests are more rapid but are geared towards individual patient testing and do not assess environmental airborne infection risk.

TABLE 1
Summary of results

| Number of samples | 25 | |
|---|---|---|
| analyzed (Phase I without VTM) | | |
| Found positive using Protein ELISA | 1 | (4% positive) |
| Found positive using Lanthanide Array | 5 | (20% positive) |
| Found positive using RT-LAMP | Not detected | |
| Found positive using RT-PCR | Not detected | |
| Number of samples | 8 | |
| analyzed (Phase II with VTM) | | |
| Found positive using Protein ELISA | 5 | (62.5% positive) |

| | |
|---|---|
| Found positive using RT-LAMP | Not detected |
| Found positive using RT-PCR | Not detected |

Note:
Condensate samples collected during Phase I and Phase II samples included viral transport medium (VTM) in tank to stabilize any collected virus. RT-LAMP and RT-PCR analyses were performed on RNA isolated from samples; ELISA and Lanthanide array were performed directly on the samples. Abbreviations: ELISA, enzyme-linked immunosorbent assay; RT-PCR, reverse-transcription polymerase chain reaction; VTM, viral transport medium.

Sampling in the current testing was done at regular intervals of 24-72 h and the condensate stored at 4° C. for further analyses. To ascertain the stability of the viral RNA, VTM was used for the latter phase of studies while maintaining the same sampling intervals. RNA was detected in both phases of the testing although the presence of SARS-CoV-2 viral RNA was not confirmed using both RT-PCR and RT-LAMP. As stated above, this could be due to the extensive dilution of the viral RNA in the sample chamber and the limitations of the detection methods for wastewater samples. It is believed that the inherent instability of the viral RNA in different processing steps might not be the reason behind this failure in SARS-CoV-2 detection as is also supported by the recently published reports. The viral RNA remains detectable and does not degrade for up to 7 days or longer in VTM (Rogers et al., 2020). In fact, stability studies of the influenza virus A (H1N1) in a similar storage medium (PrimeStore MTM) indicate that viral RNA can be preserved and stabilized for up to 30 days under these conditions (Daum et al., 2011). Since the Coronavirus is an enveloped virus, its recovery rate from water samples is substantially lower than that of non-enveloped viruses (Rusinol et al., 2020). The major approaches to concentrate water samples include precipitation using polyethylene glycol (PEG), adsorption/elution, centrifugal ultrafiltration, aluminum hydroxide flocculation, and electronegative filtration (Ahmed et al., 2020; Hjelmsø et al., 2017). Recovery rates are also specific to the strain of the virus, their charge and hydrophobicity, and partition to solids. Notably, the results shown herein provides a novel method for air sampling in any resource-limited settings across the globe. Coupled with sensitive and rapid assays that are being developed, there is the possibility of achieving near real-time sensing of SARS-CoV-2 in the atmosphere, thereby providing an actionable threat assessment.

Although RT-LAMP and RT-PCR-based analyses did not detect the virus, as mentioned earlier this may be attributed to the dilution of the viral concentration in a large volume of media and inherent instability of the viral RNA in the further processing steps used. In support of this conclusion, past studies on wastewater sampling and detection indicate the low concentration of the virus to be a major limitation (La Rosa et al., 2020). The key to the present invention is the ability to reliably integrate air sampling, virus capture, virus concentration, virus detection, and virus confirmation. By capturing virus from a known volume of air (specified by the room dimensions) and then measuring the amount of virus, it is possible to determine the viral load and thereby assess infection risk in the hospital environment.

There are three important parameters for this process: (1) the flow rate of air through the sampler; (2) the sampler run time; and (3) the amount of virus collected. The volume of air is simply calculated by multiplying the flow rate through the sampler by the sampler run time. However, the capture efficiency is a function of not only the viral load but temperature, and humidity parameters in the sampling environment, in which case inferring the original amount of virus in the air from the amount of virus captured may be a source of a significant error in the method.

RT-PCR has a limit of detection (LOD) of 6 copies/μl while RT-LAMP has a corresponding value of 0.75 copies/W. It may be presumed that these LOD values are above the detection limit required for analyses of the wastewater samples used here where the viruses are extensively diluted. Typical limits of detection required for wastewater analyses are in the range of 2 copies/100 ml-3×10³ copies/ml (Foladori et al., 2020). In the current testing, details concerning the persons in the hospital near samplers were not accessed. Instead, the focus of the present invention was on environmental monitoring of the viral load in different locations. In addition, any SARS-CoV-2 infected patients were possibly on closed-circuit ventilators, and the efficiency of air exchanges in different locations of the hospital also varied.

Further testing is conducted on aerosol collection from subjects in a defined area to confirm the viral load in those samples. Droplets naturally emanating from humans during respiration, speech and cough contain epithelial cells and immune system cells, inorganic ions (sodium, potassium, and chloride) present in mucous and saliva, and infectious load (bacteria, fungi, and virus). On the other hand, the droplets generated artificially in hospital settings have sterile water containing saline and pharmaceutical aerosols as the primary constituents. These factors should be taken into account (Atkinson et al., 2009).

The testing methods of the present invention were further validated using the ability of the dehumidifier to collect aerosolized sodium chloride. The results for the validation study on the collection of aerosolized substances from the air are shown in FIG. 4. It can be seen that between the time sodium chloride was added to the water inside the humidifier and the time the humidifier was turned off, the conductivity of the condensate collected inside the dehumidifier increases with the humidity inside the test room. The correlation coefficient of the two variables was calculated to be 0.985, demonstrating that the dehumidifier is capable of collecting aerosolized components.

In light of the recent pandemic, most countries are struggling to strike a balance between protecting their residents and maintaining their economies. In such unprecedented times, the world has witnessed overburdening of healthcare facilities and increased risk of transmission via healthcare workers and in places with high human footfall. In an attempt to reduce the possibility of infection by adopting testing methods capable of producing effective and fast results in a cost-effective manner, the present invention provides a simple, facile, and affordable testing method for areas with high population density or footfall by avoiding laborious and time-consuming individual testing. The use of dehumidifiers in designated areas would allow for analysis of the collected condensate in a rapid and facile manner, thus allowing authorities to designate zones as “hot spots” in case of a positive result. The method of sampling is both novel and effective, given the nature of transmission of coronaviruses and the unavailability of individual testing in many remote areas.

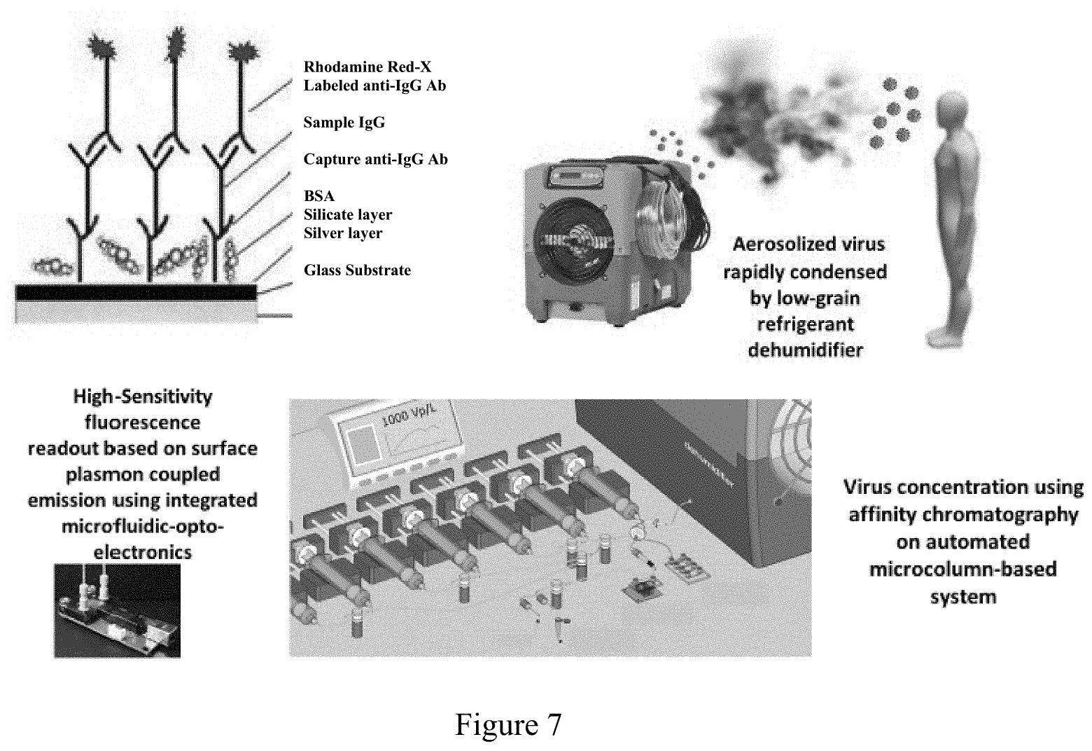

The present invention contemplates a system that can provide an output for determining the level of a virus components in a short time, such as between 10 and 30 minutes. The method and quick return system are shown in FIG. 7 and includes a system referred to as Bio-Mod as described in WO2020/068173, U.S. Pat. Nos. 9,388,373; 9,982,227 and 10,774,304 (the contents of which are incorporated by reference herein for all purposes) which is adapted for SARS-CoV-2 concentration and detection. The hardware is designed in two parts with fixed hardware (pumps, sensor, tablet computer) and a single-use bioprocess train (reactor, syringes, tubing, microfluidic mixers, capture and polishing columns). An end-to-end integrated system is built using a rapid prototyping approach wherein the Bio-Mod system is integrated with the dehumidifier. For initial testing of the integrated system, to ensure safe yet rapid method development, a non-infectious source is employed for surrogates or mimics of SARS-CoV-2. Notably, SARS-CoV-2 mimics AG-V19 from AscentGene and GenTarget that express spike protein and can be used as a non-infectious surrogate to safely develop the integrated system.

In the integrated Bio-Mod system, the condensate (which contains the collected bioaerosols) is combined with buffer and this mixture is then directed to the capture microcolumn, whose packing consist of particles (HisPur™ Cobalt Resin, tentacled particles, CaptoCore 700 etc.). These particles have specific antibodies bound to the surface such that any SARS-CoV-2 antigens present in the sample will be captured. As a first pass, commercially available anti-SARS-CoV-2 antibody conjugated to various resins as the capture matrix is used. As an alternative capture matrix, histidine tagged Griffithsin may be used captured on a metal affinity column, which is a small protein reported to bind SARS-CoV-2 and many other viruses with high affinity.

During the testing, a second buffer containing labeled secondary anti-SARS-CoV-2 spike protein antibody flows through the column, then washed to remove unbound labeled antibody. The amount of remaining labelled antibody is read directly on-column using a fluorescence detector as shown in FIG. 7. For example, surface-plasmon coupled emission detection may be used, which has the potential to detect antibody binding at diffusion-controlled rates and may a five-minute detection time. Other methods may be used, such as, gold nano-particle electrochemical detector for measuring virus antigen using an electrochemical scheme demonstrated in the Pan lab (Alafeef, M, Dighe, K. 2020) or a virus antigen-bound label to be detected on-column with an optical detector that is mounted past the column. The integrated system involves a few steps, such as Step 1: Rapidly condense aerosolized virus using low grain refrigerant dehumidifier; Step 2: Concentrate virus in condensate with affinity microcolumn; Step 3: Rapid flow ELISA detection; Step 4: Regenerate microcolumn for next sample; and Step 5. Recover sample for further analysis.

If the sample is positive, the sample is flagged, and system sends an alert that the sample needs to go for confirmatory testing. Then, the positive sample is heat inactivated and stored for retrieval. If negative, sample goes to waste drain.

This entire process is estimated to take approximately 15 minutes from end-to-end with the results displayed on the device at the end of this period. Notably in fifteen minutes, the column can be regenerated and ready for the next sample. By using two multiplexed columns, one can use them alternately and obtain readouts every 15 minutes as specified. If more frequent sampling is desired, one can increase the number of columns to obtain a higher density readout. In addition to their use as single-use elements in the Bio-MOD systems, microcolumn-based sensors have been developed that use immobilized binding proteins.

### REFERENCES

The contents of all references cited herein are incorporated herein by reference for all purposes.
Ahmed, W., Bertsch, P. M., Bivins, A., Bibby, K., Farkas, K., Gathercole, A., & Kitajima, M. (2020). Comparison of virus concentration methods for the RT-qPCR-based recovery of murine hepatitis virus, a surrogate for SARS-CoV-2 from untreated wastewater. Science of the Total Environment, 739, 139960.
Alafeef, M., Dighe, K., & Pan, D. (2019). Label-free pathogen detection based on yttrium-doped carbon nanoparticles up to single-cell resolution. ACS Applied Materials & Interfaces, 11(46), 42943-42955.
Alafeef, M, Dighe, K., Moitra, P., Pan, D. Rapid, Ultrasensitive, and Quantitative Detection of SARS-CoV-2 Using Antisense Oligonucleotides Directed Electrochemical Biosensor Chip. ACS Nano 2020, 14, 12, 17028-17045.
Alafeef, M., Moitra, P., & Pan, D. (2020). Nano-enabled sensing approaches for pathogenic bacterial detection. Biosensors and Bioelectronics, 165, 112276.
Atkinson, J., Chartier, Y., Pessoa-Silva, C. L., Jensen, P., Li, Y., & Seto, W. (2009). Natural ventilation for infection control in health-care settings. World Health Organization.
Chia, P. Y., Coleman, K. K., Tan, Y. K., Ong, S. W. X, Gum, M., Lau, S. K., Lim, X. F., Lim, A., S., Sutjipto, S., Lee, P. H., Son, T. T., Young, B. E., Milton, D. K., Gray, G. C., Schuster, S., Barkham, T., De, P. P., Vasoo, S., Chan, M., Ang, B. S. P., Tan, B. H., Leo, Y.-S., Ng, O.-T., Wong, M. S. Y., & Marimuthu, K. (2020). Detection of air and surface contamination by SARS-CoV-2 in hospital rooms of infected patients. Nature Communications, 11(1), 1-7.
Daum, L., Worthy, S., Yim, K., Nogueras, M., Schuman, R., Choi, Y., & Fischer, G. W. (2011). A clinical specimen collection and transport medium for molecular diagnostic and genomic applications. Epidemiology and Infection, 139(11), 1764-1773.
Foladori, P., Cutrupi, F., Segata, N., Manara, S., Pinto, F., Malpei, F., Bruni, L., & La Rosa, G. (2020). SARS-CoV-2 from faeces to wastewater treatment: What do we know? A review. Science of The Total Environment, 743, 140444.
Furukawa, N. W., Brooks, J. T., & Sobel, J. (2020). Evidence supporting transmission of severe acute respiratory syndrome coronavirus 2 while presymptomatic or asymptomatic. Emerging Infectious Diseases, 26(7), e1-e6.
Guo, Z.-D., Wang, Z.-Y., Zhang, S.-F., Li, X., Li, L., Li, C., & Chi, X.-Y. (2020). Aerosol and surface distribution of severe acute respiratory syndrome coronavirus 2 in hospital wards, Wuhan, China, 2020. Emerging Infectious Diseases, 26(7), 1583-1591.
Hamner, L. (2020). High SARS-CoV-2 attack rate following exposure at a choir practice-Skagit County, Washington, March 2020. Morbidity and Mortality Weekly Report, 69(19), 606-610.
Hjelmsø, M. H., Hellmér, M., Fernandez-Cassi, X., Timoneda, N., Lukjancenko, O., Seidel, M., & Schultz, A. C. (2017). Evaluation of methods for the concentration and extraction of viruses from sewage in the context of metagenomic sequencing. PLoS One, 12(1), e0170199.
Lednicky, J. A., Lauzardo, M., Fan, Z. H., Jutla, A., Tilly, T. B., Gangwar, M., & Eiguren-Fernandez, A. (2020). Viable SARS-CoV-2 in the air of a hospital room with COVID-19 patients. International Journal of Infectious Diseases, 100, 476-482.
Lee, S., Meyler, P., Mozel, M., Tauh, T., & Merchant, R. (2020). Asymptomatic carriage and transmission of SARS-CoV-2: What do we know? [Patients asymptomatiques du SARS-CoV-2 et transmission du virus: Oú en sont nos connaissances?]. Canadian Journal of Anaesthesia/Journal Canadien d' Anesthesie, 67(10), 1424-1430.
Lewis, D. (2020). Is the coronavirus airborne? Experts can't agree. Nature, 580(7802), 175.
Liu, Y., Ning, Z., Chen, Y., Guo, M., Liu, Y., Gali, N. K., & Westerdahl, D. (2020). Aerodynamic analysis of SARS-CoV-2 in two Wuhan hospitals. Nature, 582(7813), 557-560.
Lu, J., Gu, J., Li, K., Xu, C., Su, W., Lai, Z., & Yang, Z. (2020). COVID-19 outbreak associated with air conditioning in restaurant, Guangzhou, China, 2020. Emerging Infectious Diseases, 26(7), 1628-1631.
Ma, J., Qi, X., Chen, H., Li, X., Zhan, Z., Wang, H., & Morawska, L. (2020). Exhaled breath is a significant source of SARS-CoV-2 emission. medRxiv.
Moitra, P., Alafeef, M., Dighe, K., Frieman, M. B., Pan, D. (2020). Selective naked-eye detection of SARS-CoV-2 mediated by N gene targeted antisense oligonucleotide capped plasmonic nanoparticles. ACS Nano, 14, 7617-7627.
Moitra, P., Subramanian, Y., & Bhattacharya, S. (2017). Concentration dependent self-assembly of TrK-NGF receptor derived tripeptide: new insights from experiment and computer simulations. The Journal of Physical Chemistry B, 121(4), 815-824.
Morawska, L., & Cao, J. (2020). Airborne transmission of SARS-CoV-2: The world should face the reality. Environment International, 139, 105730.
Morawska, L., Tang, J. W., Bahnfleth, W., Bluyssen, P. M., Boerstra, A., Buonanno, G., & Franchimon, F. (2020). How can airborne transmission of COVID-19 indoors be minimised? Environment International, 142, 105832-105837.
Pekosz, A., Parvu, V., Li, M., Andrews, J. C., Manabe, Y. C., Kodsi, S., Gary, D. S., Roger-Dalbert, C., Leitch, J., & Cooper, C. K. (2021). Antigen-based testing but not real-time polymerase chain reaction correlates with severe acute respiratory syndrome coronavirus 2 viral culture. Clinical Infectious Diseases, ciaa1706.
Prather, K. A., Wang, C. C., & Schooley, R. T. (2020). Reducing transmission of SARS-COV-2. Science, 368(6498), 1422-1424.
Prussin, A. J., & Marr, L. C. (2015). Sources of airborne microorganisms in the built environment. Microbiome, 3(1), 78.
Raissi, M., Perdikaris, P., & Karniadakis, G. E. (2019). Physics-informed neural networks: A deep learning framework for solving forward and inverse problems involving nonlinear partial differential equations. Journal of Computational Physics, 378, 686-707.
Rogers, A. A., Baumann, R. E., Borillo, G. A., Kagan, R. M., Batterman, H. J., Galdzicka, M. M., & Marlowe, E. M. (2020). Evaluation of transport media and specimen transport conditions for the detection of SARS-CoV-2 by use of real-time reverse transcription-PCR. Journal of Clinical Microbiology, 58(8), 1-5.
La Rosa, G., Iaconelli, M., Mancini, P., Bonanno Ferraro, G., Veneri, C., Bonadonna, L., & Suffredini, E. (2020). First detection of SARS-COV-2 in untreated wastewaters in Italy. Science of the Total Environment, 736, 139652.
Rusiñol, M., Martinez-Puchol, S., Forés, E., Itarte, M., Girones, R., & Bofill-Mas, S. (2020). Concentration methods for the quantification of coronavirus and other potentially pandemic enveloped virus from wastewater. Current Opinion in Environmental Science & Health, 17, 21-28.
Santarpia, J. L., Herrera, V. L., Rivera, D. N., Ratnesar-Shumate, S., Denton, P. W., Martens, J. W. S., & Lawler, J. V. (2020). The infectious nature of patient-generated SARS-CoV-2 aerosol. MedRxiv.
Santarpia, J. L., Rivera, D. N., Herrera, V. L., Morwitzer, M. J., Creager, H. M., Santarpia, G. W., & Broadhurst, M. J. (2020). Aerosol and surface contamination of SARS-CoV-2 observed in quarantine and isolation care. Scientific Reports, 10(1), 1-8.
Stadnytskyi, V., Bax, C. E., Bax, A., & Anfinrud, P. (2020). The airborne lifetime of small speech droplets and their potential importance in SARS-CoV-2 transmission. Proceedings of the National Academy of Sciences of the United States of America, 117(22), 11875-11877.
WHO. (2020). Modes of transmission of virus causing COVID-19: implications for IPC precaution recommendations: scientific brief. WHO reference number: WHO/2019-nCoV/Sci_Brief/Transmission_modes/2020.2.

TABLE 2
Concentration of SARS-CoV-2 S-protein
as determined by the ELISA assay.

| Sample Number | Concentration (ng/mL) |
|---|---|
| 0630_4 | 0.52 |
| 0630_3 | 0.56 |
| 0701_3 | Not detected |
| 0701_4 | 0.41 |
| 0702_4 | 0.55 |
| 0702_3 | 0.43 |
| 0703_3 | 0.56 |
| 0703_4 | 0.59 |
| 0630_1 | 0.26 |
| 0630_2 | 0.37 |
| 0701_2 | 0.15 |
| 0701_1 | 0.55 |
| 0702_1 | 0.42 |
| 0702_2 | Not detected |
| 0703_2 | 0.37 |
| 0703_1 | 0.63 |
| 0706_2b | 0.50 |
| 0706_4 | 0.91 |
| 0706_1d | 0.85 |
| 0706_1a | 0.94 |
| 0706_1b | 0.80 |
| 0706_1c | 1.13 |
| 0706_3 | 1.36 |
| 0706_2c | 2.61 |
| 0706_2a | 0.67 |

Note:
The sample code starts with the date of sample collection from hospital followed by the dehumidifier number, i.e., 0630_4 indicates the water sample has been collected from dehumidifier number 4.

TABLE 3
Comparison of lanthanide-doped carbon nanoparticles
sensor array results with Spike protein ELISA.

| | LCNPs biosensor | Results based on our sensor |
|---|---|---|
| Sample Number | (ΔI/I₀) | (15 minutes) |
| 0630_4 | 0.43 | −VE |
| 0630_3 | 0.49 | −VE |
| 0701_3 | 0.46 | −VE |
| 0701_4 | 0.55 | −VE |
| 0702_4 | 0.47 | −VE |
| 0702_3 | 0.51 | −VE |
| 0703_3 | 0.52 | −VE |
| 0703_4 | 0.48 | −VE |
| 0630_1 | 0.30 | −VE |
| 0630_2 | 0.39 | −VE |
| 0701_2 | 0.26 | −VE |
| 0701_1 | 0.29 | −VE |
| 0702_1 | 0.33 | −VE |
| 0702_2 | 0.31 | −VE |
| 0703_2 | 0.47 | −VE |
| 0703_1 | 0.47 | −VE |
| 0706_2b | 0.76 | −VE |
| 0706_4 | 1.40 | −VE |
| 0706_1d | 1.66 | −VE |
| 0706_1a | 2.38 | +VE |
| 0706_1b | 2.97 | +VE |
| 0706_1c | 2.65 | +VE |
| 0706_3 | 2.18 | +VE |
| 0706_2c | 2.37 | +VE |
| 0706_2a | 1.95 | −VE |

Note:
The sample code starts with the date of sample collection from the hospital followed by the dehumidifier number, i.e., 0630_4 indicates the water sample has been collected from dehumidifier number 4.

TABLE 4
Summarization of RNA extraction results for the sampling

| Sample | Concentration | | | |
|---|---|---|---|---|
| Number | (ng/μL) | A₂₆₀/A₂₈₀ | A₂₆₀/A₂₃₀ | Inference |
| 0723_1a | — | — | — | No RNA detected |
| 0723_2a | — | — | — | No RNA detected |
| 0723_3a | — | — | — | No RNA detected |
| 0723_4a | — | — | — | No RNA detected |
| 0728_1a | 0.4 | 1.23 | 0.09 | RNA detected |
| 0728_2a | 0.3 | 1.8 | 0.07 | RNA detected |
| 0728_3a | 0.4 | 1.39 | 0.19 | RNA detected |
| 0728_4a | 0.5 | 1.50 | 0.05 | RNA detected |
| 0730_1a | — | — | — | No RNA detected |
| 0730_2a | — | — | — | No RNA detected |
| 0730_3a | — | — | — | No RNA detected |
| 0730_4a | — | — | — | No RNA detected |
| 0803_1a | — | — | — | No RNA detected |
| 0803_2a | 0.1 | 0.57 | 0.01 | RNA detected |
| 0803_3 | 2.7 | 1.36 | 0.25 | RNA detected |
| 0803_4 | 0.3 | 0.33 | 0.01 | RNA detected |
| 0807_1a | 1.3 | 1.72 | 0.01 | RNA detected |
| 0807_2a | 0.3 | 0.57 | 0.06 | RNA detected |
| 0807_3a | 0.6 | 0.79 | 0.02 | RNA detected |
| 0807_4 | 0.2 | 0.37 | 0.00 | RNA detected |
| 0810_1a | 0.2 | 0.24 | 0.08 | RNA detected |
| 0810_2a | 0.2 | 0.32 | 0.03 | RNA detected |
| 0810_3a | — | — | — | No RNA detected |
| 0810_4 | — | — | — | No RNA detected |

Note:
The sample code starts with the date of sample collection from hospital followed by the dehumidifier number, i.e., 0723_1a indicates the water sample has been collected from dehumidifier number 1.

TABLE 5
Results of RNA extraction from samples collected in VTM.

| Sample | | | | |
|---|---|---|---|---|
| Collection | | | | |
| Date, | Concentration | | | |
| Number | (ng/μL) | A₂₆₀/A₂₈₀ | A₂₆₀/A₂₃₀ | Inference |
| 0903_1a | — | — | — | No RNA |
| | | | | detected |
| 0903_2a | 6.2 | 1.52 | 0.03 | RNA detected |
| 0903_3a | — | — | — | No RNA |
| | | | | detected |
| 0903_4a | 0.1 | 0.24 | 0.0 | RNA detected |
| 0910_1a | 2.0 | 1.48 | 0.14 | RNA detected |
| 0910_2a | 12.5 | 1.51 | 0.58 | RNA detected |
| 0910_3a | 1.5 | 1.15 | 0.05 | RNA detected |
| 0910_4a | 0.9 | 0.67 | 0.15 | RNA detected |

Note:
The sample code starts with the date of sample collection from hospital followed by the dehumidifier number, i.e., 0903_1a indicates the water sample has been collected from dehumidifier number 1.

That which is claimed:

1. A method of collecting bioaerosols particles of a suspected virus in a defined area, the method comprising:
positioning a dehumidifier in the defined area;
collecting the bioaerosols particles in the condensate of the dehumidifier; and
concentrating the condensate to isolate any captured bioaerosols particles for further analysis.

2. The method of claim 1, further comprising removing condensate from the dehumidifier prior to concentration of said condensate.

3. The method of claim 1, wherein the bioaerosols particles comprise virus particles or biomarkers.

4. The method of claim 1, further comprising analyzing the captured bioaerosols particles for coronavirus biomarkers selected from COVID-19 or mutants or variants thereof.

5. The method of claim 1, wherein the collecting of the bioaerosols particles in the condensate is for about 10 minutes to several days.

6. The method of claim 1, wherein the bioaerosols particles comprise virus particles or biomarkers and wherein the captured virus particles or biomarkers are analyzed to determine virus type and quantity.

7. The method of claim 1, wherein the dehumidifier is a low-grain refrigerant (LGR) dehumidifier.

8. The method of claim 1, wherein concentration of the condensate to isolate virus particles or biomarkers in the condensate is effectuated using an affinity microcolumn.

9. The method of claim 1, wherein the bioaerosols particles comprise virus particles or biomarkers and wherein the captured virus particles or biomarkers are analyzed using rapid flow enzyme-linked immunosorbent assay (ELISA).

10. The method of claim 1, wherein collecting the bioaerosols particles in the condensate of the dehumidifier is conducted under humidity conditions ranging from about 40-60% at about room temperature.

11. The method of claim 1, wherein the bioaerosols particles comprise virus particles or biomarkers and wherein the captured virus particles or biomarkers are analyzed using RNA based analysis employing commercially available RT-LAMP, reverse-transcription polymerase chain reaction (RT-PCR) kits, or a nano-sensing platform using lanthanide-doped carbon nanoparticles (LCNPs), to provide a distinct fluorescence response in the presence of SARS-CoV-2.

12. The method of claim 1, wherein the dehumidifier further comprises viral transport medium (VTM) in a condensate container to stabilize any collected virus.

13. The method of claim 1, wherein the concentrated condensate is analyzed for RNA or S-protein from COVID-19 or mutants or variants thereof.

14. The method of claim 1, wherein a humidifier is positioned in the defined area to increase moisture content in the defined area.

15. A system for detecting aerosolized virus particles or biomarkers in an atmosphere within a defined space, the system comprising:
a dehumidifier for collecting the aerosolized virus particles or biomarkers, wherein collected aerosolized virus particles or biomarkers are contained in a condensate of the dehumidifier;
a collection system for removing the condensate from the dehumidifier;
an affinity microcolumn for concentrating collected aerosolized virus particles or biomarkers, wherein the collection system is communicatively connected to the affinity microcolumn; and
a detection system for analyzing the collected aerosolized virus particles or biomarkers in the condensate.

16. The system of claim 15, wherein the dehumidifier is a low-grain refrigerant (LGR) dehumidifier.

17. The system of claim 15, wherein the detection system comprises a rapid flow enzyme-linked immunosorbent assay (ELISA).

18. The system of claim 15, wherein collecting the aerosolized virus particles or biomarkers in the condensate of the dehumidifier is conducted under humidity conditions ranging from about 40-60% at room temperature.

19. The system of claim 15, wherein the detection system comprises a RNA based analysis device employing commercially available RT-LAMP. reverse-transcription polymerase chain reaction (RT-PCR) system, or a nano-sensing platform using lanthanide-doped carbon nanoparticles (LCNPs).

20. The system of claim 15, wherein the dehumidifier further comprises viral transport medium (VTM) in a condensate container to stabilize any collected virus.
